(19) United States
(12) Patent Application Publication
(10) Pub. No.: US 2004/0010241 A1
(43) Pub. Date: Jan. 15, 2004

(54) Absorbent article having elasticized regions providing improved comfort

(75) Inventors: Donald Joseph Sanders, Larsen, WI (US); Paul VanGompel, Hortonville, WI (US); Jennifer Lynn Marvin, Greenville, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/196,112

(22) Filed: Jul. 15, 2002

(51) Int. Cl. A61F 13/15; A61F 13/20

(52) U.S. Cl. 604/385.24

(57) ABSTRACT

It has been found that an absorbent article having improved fit at the waist region can be made by changing the stretch properties about the waist region such that the sides stretch more than the front or back. One method of performing this function is to change the modulus of elasticity of the side regions relative to the front and back regions. For instance, by use of more elastic or stronger elastic in the front and back of the waist relative to the side of the waist, the modulus of elasticity in the front and back will be higher than at the sides of the absorbent article. If the same tension is applied uniformly to such a waist, the sides will stretch more than the front or back. This improves the absorbent article's fit because the hips of the body can curve more than the front or back waist portion, which can be relatively flat. By having the greatest stretch at the sides, they can conform to the body's increased curvature at the hips.

### BACKGROUND OF THE INVENTION

Disposable absorbent articles are used by babies, toddlers, children, and incontinent adults to absorb and contain bodily wastes. Frequently, these absorbent articles are provided with a waist opening, containing one or more elastic members. The elastic members are intended to maintain the position of the absorbent article while being worn and to promote a comfortable fit. Generally, the absorbent articles are provided with an elasticized waist opening having the same stretch properties at all points within the waist region. Thus, the fit or stretch of the waist is the same at the front, back, and side waist portions.

However, it is known that a person's waist is not a perfect circle, and that the waist can be more elliptical in shape than round. Thus, the side waist portions of an absorbent article must stretch more than the front or back waist portions, since frequently the body's point of greatest curvature can be at the hips and not in the front or back. By stretching more at the sides and less in the front or back, the absorbent article will have better conformance to the body. Existing absorbent articles having unitary stretch properties do not perform this function well. Therefore, what is needed is an improved absorbent article having a waist portion that provides differential stretch properties between the front, back, and side waist portions such that the article possesses an improved fit around the waist.

### SUMMARY OF THE INVENTION

It has been found that an absorbent article having an improved fit at the waist region can be made by changing the stretch properties about the waist region such that the sides stretch more than the front or back. One method of performing this function is to change the modulus of elasticity of the side regions relative to the front or back regions. For instance, by placing twice as much elastic in the front and back waist regions relative to the side waist regions, the modulus of elasticity in the front and back will be higher than at the sides of the waist. Furthermore, if the same tension is applied uniformly to such a waist region, the sides will stretch more than the front or back.

In one aspect, the invention resides in an absorbent article including a front portion having a front center region, a pair of front side regions located adjacent the front center region and a pair of front side edges. The front center and the front side regions each have a plurality of zigzag elastic members, wherein the stretch of the front center region is less than the stretch of the front side elastic regions when subjected to the same tensile force. The absorbent article also includes a back portion having a pair of back side edges, and a crotch portion having an absorbent structure connecting the front and back portions. A bottomsheet is secured to at least one of the front, back, or crotch portions.

In another aspect, the invention resides in an absorbent article including a front portion having a front center region, a pair of front side regions located adjacent the front center region, and a pair of front side edges, the front center and the front side regions each having a plurality of zigzag elastic members, wherein the stretch of the front center region is less than the stretch of the front side regions when subjected to the same tensile force. The absorbent article also includes a back portion having a back center region, a pair of back side regions located adjacent the back center region, and a pair of back side edges, the back center and the back side regions each having a plurality of zigzag elastic members, wherein the stretch of the back center region is less than the stretch of the back side regions when subjected to the same tensile force. The absorbent article further includes a crotch portion having an absorbent structure connecting the front and back portions; a bottomsheet secured to at least one of the front, back, or crotch portions; and the front side edges joined to the back side edges forming an underwear like absorbent article having a waist opening and a pair of leg openings.

In yet another aspect, the invention resides in an absorbent article including a front portion having a front center region, a pair of front side regions located adjacent the front center region, and a pair of front side edges, the front center and the front side regions each having a plurality of zigzag elastic members, wherein the stretch of the front center region is less than the stretch of the front side elastic regions when subjected to the same tensile force. The absorbent article also includes a back portion having a back center region, a pair of back side regions located adjacent the back center region, and a pair of back side edges, the back center and the back side regions each having a plurality of zigzag elastic members, wherein the stretch of the back center region is less than the stretch of the back side regions when subjected to the same tensile force. The absorbent article further includes a crotch portion having an absorbent structure connecting the front and back portions; a bottomsheet secured to at least one of the front, back, or crotch portions; and the front side edges connected to the back side edges by a pair of refastenable members and by a pair of semi-permanent bonds forming an underwear like absorbent article having a waist opening and a pair of leg openings, and wherein the absorbent article is capable of being adjusted or removed by breaking the semi-permanent bonds.

### BRIEF DESCRIPTION OF THE DRAWINGS

The foregoing aspects of the invention, and other features, aspects, and advantages of the invention will become better understood with regard to the following description, appended claims, and accompanying drawings where:

FIG. 1 illustrates a plan view of a partially cut away absorbent article according to one embodiment of the present invention.

FIG. 2 illustrates a plan view of one embodiment for the elastic members of the present invention.

FIG. 3 illustrates a plan view of another embodiment for the elastic members of the present invention.

FIG. 4 illustrates a plan view of another embodiment for the elastic members of the present invention.

### DEFINITIONS

As used herein, “joined or joining” includes configurations where one element is directly or indirectly attached to another element by any means including, but not limited to, adhesives, thermal bonding, sonic bonding, chemical bonding, mechanical bonding, pressure bonding, heat and pressure bonding, hydrogen bonding, fasteners, stitching, or other means known to those of skill in the art. Joined also includes configurations where elements are indirectly joined together. By “indirectly joined” it is meant one element is attached to a second element by one or more intermediate layers. For instance, the outer layers in an ordinary plywood laminate are indirectly joined to each other by the laminate's other layers.

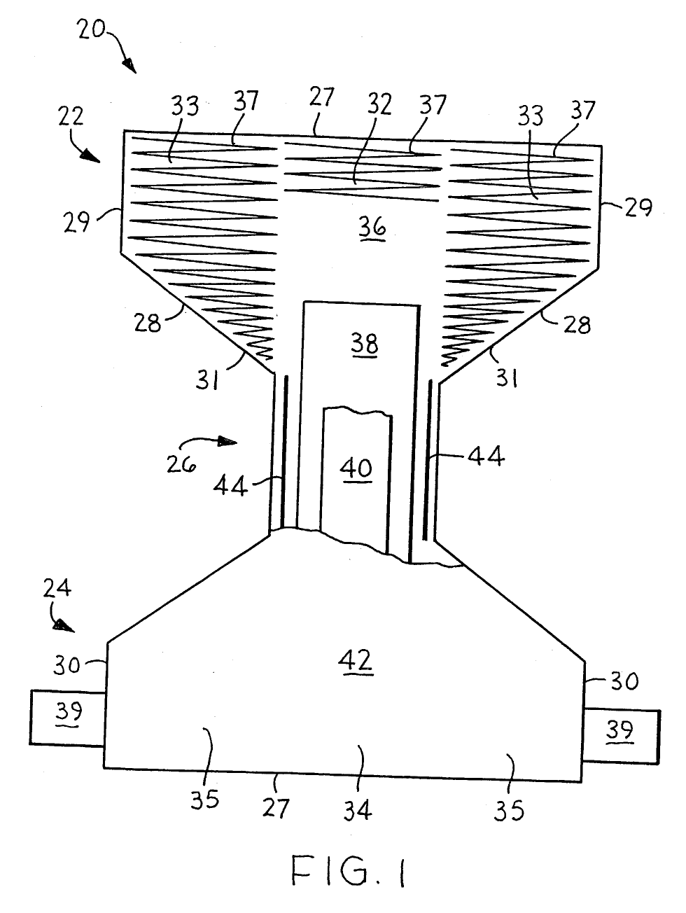

As used herein, “zigzag elastic members” means an elastic material that is applied to the substrate such that consecutive elastic members are non-parallel to each other. The zigzag elastic members may be formed from a continuous elastic material or formed from discrete elastic elements applied to the substrate. In addition, the zigzag elastic members can be linear or they can contain loops or other curvilinear portions. A common form for the zigzag elastic members is obtained when a continuous elastic material is applied to the substrate linearly between a plurality of alternating endpoints such that a series of alternating “V” shaped portions of the elastic material is formed as illustrated in FIG. 1, but the invention is not limited exclusively to this form.

### DETAILED DESCRIPTION OF THE INVENTION

Referring to FIG. 1, a plan view of one portion of an absorbent article 20 is shown in a flat uncontracted state. Portions of the absorbent article 20 are shown partially cut away to show interior construction details. The absorbent article 20 includes a front portion 22, a back portion 24, and a crotch portion 26 joining the front and back portions. The absorbent article also includes a pair of longitudinally opposed end edges 27, and a pair of laterally opposed side edges 28. The opposed end edges 27 define a waist opening for the absorbent article. The opposed end edges 27 are typically straight, but they can be curvilinear to improve the fit of the absorbent article.

The opposed side edges 28 contain leg openings for the absorbent article 20 and they can be tapered or curvilinear such that the width between the side edges 28 is less in the crotch portion 26 than in the front and/or back portion (22, 24). The side edges 28 include a pair of front side edges 29, a pair of back side edges 30, and a pair of leg cutouts 31 connecting the front side edges 29 to the back side edges 30.

The absorbent article can also include a pair of fasteners 39 joined to the back side edges 30. Suitable fasteners are known to those of skill in the art and can include adhesive fasteners, or hook and loop type fastening methods. Alternatively, the front and back side edges (29, 30) can be joined together to form an underwear like article with a preformed waist opening. In yet another alternative, the front and back side edges (29, 30) can be joined together, and provided with a pair of perforated lines or other semi-permanent bond and a pair of fasteners to form an underwear like article with a preformed waist opening that is also refastenable. By breaking the semi-permanent bonds and adjusting the fasteners as needed, the absorbent article is capable of being adjusted smaller or larger or quickly removed. Such a design is known to those of skill in the art as evidenced by UK Patent Application GB 2 267 024 A entitled “Disposable Diapers”, which was published on Oct. 24, 1993. As seen in the publication's FIG. 1, the diaper has a cut line 9b, in the form of intermittent cuts or perforations, adjacent bond line 8 located beneath fastening flap 6 with attached fastening spots 5 serving as the fastening means.

The front portion 22 further includes a front center region 32 and a pair of front side regions 33 adjacent the front center region. Similarly, the back portion 24 includes a back center region 34 and a pair of back side regions 35 adjacent the back center region. Each of these regions can include a plurality of zigzag elastic members 37 as will be discussed in more detail later.

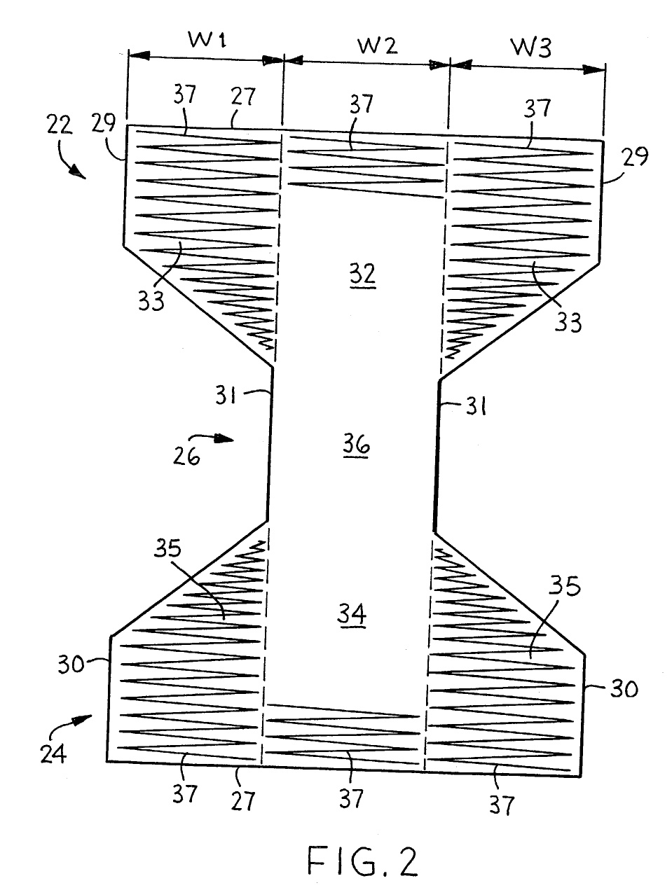

The front, back, and crotch portions (22, 24, 26) can be integral members formed from a unitary sheet of material, or they can be two or more individual members joined together forming the absorbent article 20. Similarly, the various regions forming either the front or back of the absorbent article (32, 33, 34, 35) can be integral or individual members joined together. Dashed lines are used in FIG. 2 to represent seams where individual pieces are joined together.

The absorbent article includes a bottomsheet 36 and an absorbent structure 38 disposed at least within the crotch portion 26 of the absorbent article.

The absorbent structure 38 can extend into the front center region 32, and/or extend into the front side regions 33, and/or extend into the back center region 34, and/or extend into the back side regions 35, or any combination thereof. The absorbent article 20 can also include a fluid intake/distribution layer 40, a topsheet 42, and a pair of leg elastics 44 if desired.

The various components of the absorbent article 20 are operatively assembled together employing suitable joining methods, such as adhesive, ultrasonic bonds, thermal bonds or combinations thereof. In the shown embodiment, for example, the topsheet 42 and bottomsheet 36 are assembled to each other and to the absorbent structure 38 with an adhesive, such as a hot melt adhesive. The adhesive may be applied as a uniform continuous layer of adhesive, a patterned layer of adhesive, a sprayed pattern of adhesive, or an array of separate lines, swirls or dots of adhesive. Similarly, other components, such as the elastics 37 and 44 or fasteners 39, if used, can be assembled using the above-identified joining methods.

Referring now to FIGS. 2-4 various embodiments of the zigzag elastic members 37 are illustrated in more detail. In FIGS. 2-4, only the bottomsheet 36 and the zigzag elastic members 37 are shown. The other absorbent article components have been removed for clarity. The zigzag elastic members can be attached to the bottomsheet, the topsheet, or other substrate and then incorporated into the absorbent article.

As illustrated in FIG. 2, the absorbent article has a plurality of zigzag elastic members 37 located in each of the front center, front side, back center and back side regions (32, 33, 34, 35). In this particular embodiment, the zigzag elastic members 37 are wholly contained within each specific region (32, 33, 34, 35) and do not cross into or overlap the adjacent region. While the zigzag elastic members 37 are located in both the front and back portions (22, 24), the absorbent article 20 can be constructed with the zigzag elastic members located in only the front or back portion.

The function of the zigzag elastic members 37 is to laterally contract the front and back portions (22, 24), especially along the end edges 27, to hold the absorbent article in place when worn. It has been determined that to improve the comfort and fit of the absorbent article when worn, the stretch properties for the zigzag elastic members 37 should be non-uniform. In particular, the stretch properties of the zigzag elastic members in the side regions (33, 35) should be greater than the stretch properties of the zigzag elastic members in the central regions (32, 34). Desirably, the stretch of the side regions (33, 35) is about 100 percent or greater. More desirable, the stretch of the side regions (33, 35) is about 150 percent or greater. More desirable still, the stretch of the side regions (33, 35) is about 200 percent or greater. Desirably, the stretch of the center regions (32, 34) is about 150 percent or less. More desirable, the stretch of the center regions (32, 34) is about 100 percent or less. More desirable still, the stretch of the center regions (32, 34) is about 50 percent or less.

To obtain the desired stretch property for each region, elastic members having a lower or a higher modulus of elasticity can be selected, the decitex of the elastic members can be changed, more elastic members can be applied in one region relative to another region, or the elastic members can be overlapped in the center regions. Another method to affect the stretch properties would be to make the elastics less effective by deadening them, applying more elastic adhesive in one region relative to another region, or applying the elastics at a higher tension in one region and at a lower tension in another region.

Construction of the absorbent article in this manner will promote a more comfortable garment, since the side regions (33, 35) of the absorbent article will stretch more than the center regions (32, 34) when the same tensile force is applied to each region. This improves the fit because the hips of the body can curve more than the front or back waist sections, which can be relatively flat. By having the greatest stretch at the side regions (33, 35), they can conform to the body's increased curvature at the hips. In addition, when walking or moving, the hips of the body move more than the front or back midsections. As a result, less stretch and conformance is required for these lower motion areas by the center regions (32, 34) as opposed to the hips and the side regions (33, 35). Thus, having the side regions (33, 35) stretch more than the center regions (32, 34) provides for an absorbent article that is more comfortable when walking and moving.

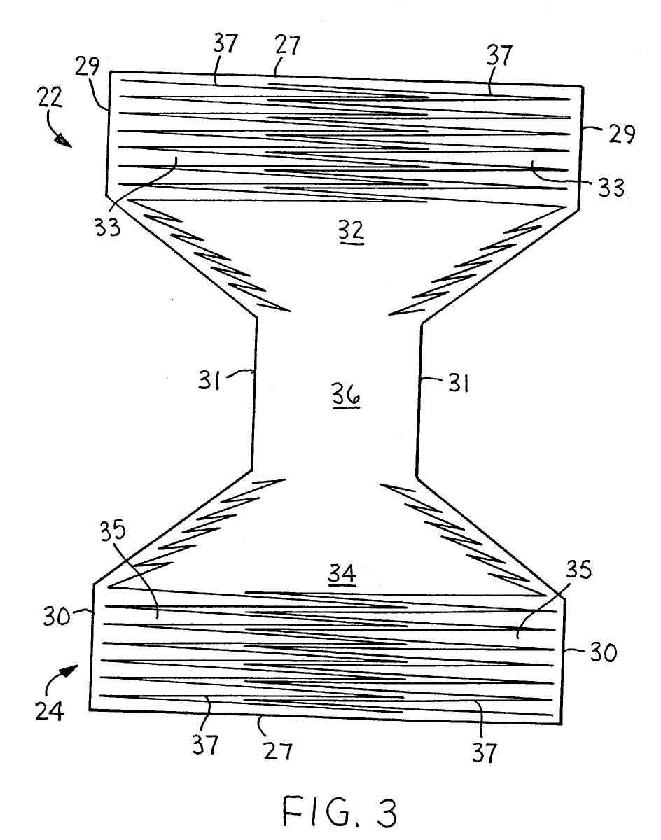

One method for determining the stretch properties of the front (or back regions) would be to clamp each of the front side edges 29 into an appropriate fixture that applies a uniform clamping force along the length of the front side edges 29, such as the jaws of a tensile tester. This would work well for the zigzag elastic members having uniform coverage as illustrated in FIG. 3. If needed, the absorbent article can be cut such that a strip of material from the waist section is tested. The width of the strip, as placed into the jaw of the tensile tester, is dependant on the configuration of the zigzag elastic members present in the absorbent article. The width should be such that the zigzag elastic members completely cover the strip from one side to the other in all three regions. For the zigzag members illustrated in FIG. 2, the test strip would be cut from an area in the front center region near end edge 27 where the zigzag elastic members are present.

The absorbent article's width measurements W1, W2, and W3 (FIG. 2) are determined in the absorbent article's relaxed position. The front center region 32 and front side regions 33 are then elongated by applying a uniform tensile force to the front side edges 29, or to the test strip having side to side coverage of the zigzag elastic members. The measurements for W1, W2, and W3 are then determined under load. The percent increase in length, relative to the initial length, should be greater for W1 and W3 than for W2. The percent increase in length is determined by the formula: ((Wₛ−Wₒ)/Wₒ)×100. Where Wₛ is the length of the waist segment in the stretched condition, and Wₒ is the length of the waist segment in the unstretched condition. Other methods known to those of skill in the art can be used to measure the stretch properties of each elasticized region on an elongation per unit width of material basis for comparison of the stretch in one region relative to another region.

The following table represents desirable design parameters for an absorbent article having side regions (33, 35) that stretch more than the center regions (32, 34). In the table the dimensions are in inches, and the waist circumference unstretched and stretched (Waistₒ, Waistₛ) is calculated based on symmetric front and back portions (22, 24) for the absorbent article.

TABLE 1
Absorbent Article Waist Designs

| Example A | W1ₒ = 5 | W2ₒ = 5 | W3ₒ = 5 | Waistₒ = 15 |
|---|---|---|---|---|
| | W1ₛ = 15 | W2ₛ = 10 | W3ₛ = 15 | Waistₛ = 40 |
| | Stretch | Stretch | Stretch | Stretch |
| | 200% | 100% | 200% | 167% |
| Example B | W1ₒ = 4 | W2ₒ = 7 | W3ₒ = 4 | Waistₒ = 15 |
| | W1ₛ = 9.75 | W2ₛ = 10.5 | W3ₛ = 9.75 | Waistₛ = 30 |
| | Stretch | Stretch | Stretch | Stretch |
| | 144% | 50% | 144% | 100% |
| Example C | W1ₒ = 2.5 | W2ₒ = 10 | W3ₒ = 2.5 | Waistₒ = 15 |
| | W1ₛ = 7.5 | W2ₛ = 20 | W3ₛ = 7.5 | Waistₛ = 35 |
| | Stretch | Stretch | Stretch | Stretch |
| | 200% | 100% | 200% | 133% |

Referring now to FIG. 3, another embodiment for the zigzag elastic members is illustrated. In this embodiment, the stretch of the center regions (32, 34) is increased by having more zigzag elastic members 37 present in the center regions (32, 34) relative to the side regions (33, 35). The increase in the number of zigzag elastic members 37 is accomplished by having the zigzag elastic members 37 from the side regions (33, 35) overlap the center regions (32, 34) resulting in twice as many elastic members 37 in the center regions (32, 34) as opposed to the side regions (33, 35). The overlapping zigzag elastic members 37 can be the only elastic members present in the center regions (32, 34) or additional elastic members can be present in the center regions.

If desired, the zigzag elastic members 37 in the side regions (33, 35) can be tapered to follow the leg cutouts 31 as shown. This has the benefit of using the zigzag elastic members 37 to provide stretch conformance at the waist, while also providing the function of leg elastics adjacent the leg cutouts 31. The zigzag elastic members 37 applied in this manner will also reduce blousy/baggy material in this area making the absorbent article more discrete when worn under clothing. If desired, the zigzag elastic members can continue through the crotch region (not illustrated) in a non-zigzag manner to provide complete elastic coverage of the leg cutout area. Alternatively, separate leg elastics 44 can be placed adjacent the leg cutouts 31 in the crotch region 26 similar to FIG. 1.

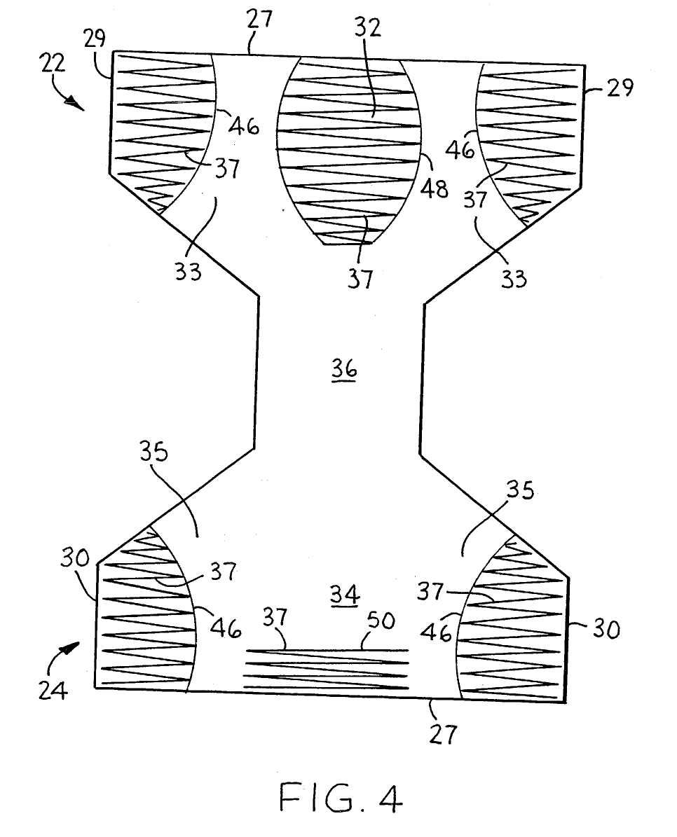

Referring now to FIG. 4, another embodiment for the zigzag elastic members 37 is illustrated. The zigzag elastic members 37 applied to the side regions (33, 35) form an arcuate curve 46 pointing towards the center regions (32, 34). The zigzag elastic members 37 in the front center region 32 are applied in an elongated oval pattern 48, while the zigzag elastic members in the back center region 34 are applied in a rectangular pattern 50. The pattern of the zigzag elastic members 37 for each region (32, 33, 34, 35) result in an absorbent article having a “control top” similar to various panty hose or girdles. Like a girdle, the absorbent article will help flatten the stomach since the front center region 32 will stretch less than the front side regions 33, and thereby hold the stomach in while allowing for stretch and conformance at the hips. Such a design provides a more desirable silhouette for women when worn, and it is especially helpful for women who may be overweight.

The zigzag elastic members 37 can be applied to the absorbent article during manufacture, such as to either the topsheet 42 or bottomsheet 36 prior to joining the respective sheet to other potions of the absorbent article. Such a process can produce the overlapped zigzag elastic members illustrated in FIG. 3 by applying one zigzag elastic member first and then applying the next zigzag elastic member over a portion of the first member. Alternatively, the entire elasticized region can be cut and formed from a substrate already containing a plurality of zigzag elastic members and then joined to the absorbent article. Such a process can be used to make the front and back side regions (33, 35) illustrated in FIG. 2, where the dashed lines represent a seam showing that the side regions are joined to the front and back center regions (32, 34).

Various suitable methods for either applying the zigzag elastic members or for making material containing the zigzag elastic members are known to those of skill in the art. For example, a method of applying a zigzag elastic member is disclosed in U.S. Pat. No. 5,000,806 entitled “Method and Apparatus for Applying an Elastic Strand to a Disposable Diaper”, which issued Mar. 19, 1991 to Merkatoris et al. and is herein incorporated by reference. Merkatoris discloses a machine having a pair of canted wheels with spindles having protruding members that nest in an S shaped configuration in one position (FIG. 4), which then stretches the elastic strands as the wheels rotate, and then applies the elastic strands to the substrate in another position (FIG. 11).

A suitable process for making an elasticized substrate having a plurality of zigzag elastic members is disclosed in U.S. Pat. No. 6,179,946 “Process for Making a Composite Sheet”, which issued Jan. 30, 2001 to Ward et al. and is herein incorporated by reference. Ward discloses a machine having a cam driven guide housing, pin conveyors, and a sheet forwarding unit as shown in FIG. 3, which applies a zigzag elastic strand to a substrate.

Any suitable material providing the desired stretch may be used for the zigzag elastic members 37. Suitable elastic strands for use as zigzag elastic members are known to those of skill in the art. For example, suitable elastic members may be composed of a 470 decitex LYCRA SPANDEX elastomer or a 620 decitex LYCRA SPANDEX elastomer commercially available from E. I. Dupont de Nemours having an office located in Wilmington, Del.

The bottomsheet 36 can be composed of a material which is either liquid permeable or liquid impermeable. It is generally preferred that the bottomsheet 36 include a liquid impermeable portion at least in the crotch region 26. The liquid impermeability of the bottomsheet can be achieved by use of a material that is liquid impermeable or by appropriate hydrophilic treatment of the bottomsheet to render it liquid impermeable by use of a coating such as latex. The bottomsheet can be manufactured from a thin plastic film or other flexible liquid impermeable material. For example, the bottomsheet may be formed from a polyethylene film having a thickness of from about 0.012 millimeter (0.5 mil) to about 0.051 millimeter (2.0 mils). If it is desired to present the bottomsheet with a more clothlike feeling, the bottomsheet can comprise a polyolefin film having a nonwoven web laminated to the outer surface thereof, such as a spunbond web of polyolefin fibers. For example, a stretch-thinned polypropylene film having a thickness of about 0.015 millimeter (0.6 mil) can have a spunbond web of polypropylene fibers thermally laminated to the film, the fibers having a thickness of about 1.5 to 2.5 denier per filament, and the nonwoven laminated web has a basis weight of about 17 grams per square meter (0.5 ounce per square yard). Methods of forming such clothlike backsheets are known to those of skill in the art.

Furthermore, the bottomsheet 36 can be a woven or nonwoven fibrous web layer which has been totally or partially constructed or treated to impart a desired level of liquid impermeability to selected regions that are adjacent or proximate the absorbent structure 38. Still further, the bottomsheet can optionally be composed of a microporous “breathable” material which permits vapors to escape from the absorbent structure 38 while still preventing liquid exudates from passing through the bottomsheet. The bottomsheet typically provides the outer cover of the absorbent article 20. The bottomsheet can also be embossed or otherwise provided with a matte finish to provide a more aesthetically pleasing appearance.

The optional topsheet 42 suitably presents a bodyfacing surface which is compliant, soft feeling, and nonirritating to the wearer's skin. The topsheet can be less hydrophilic than the absorbent structure 38, to present a relatively dry surface to the wearer, and can be sufficiently porous to be liquid permeable, permitting liquid to readily penetrate through its thickness. A suitable topsheet can be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The topsheet is suitably employed to help isolate the wearer's skin from liquids held in the absorbent structure 38.

Various woven and nonwoven fabrics can be used for the topsheet 22. For example, the topsheet may be composed of a meltblown or spunbonded web of polyolefin fibers. The topsheet may also be a bonded-carded web composed of natural and/or synthetic fibers. The topsheet may be composed of a substantially hydrophobic material, and the hydrophobic material may, optionally, be treated with a surfactant or otherwise processed to impart a desired level of wettability and hydrophilicity. The surfactant can be applied by any conventional means, such as spraying, printing, brush coating or the like. The surfactant can be applied to the entire topsheet or can be selectively applied to particular sections of the topsheet, such as the medial section along the longitudinal centerline of the absorbent article, to provide greater wettability of such sections.

The absorbent structure 38 is a fluid acquisition, storage, and distribution medium that can comprise any absorbent material such as natural or synthetic fibers, foams, or high-absorbency materials. The absorbent structure can contain one or more layers of absorbent material. A common absorbent structure includes one or more layers of a matrix of hydrophilic fibers, such as a web of cellulosic fluff, mixed with particles of a high-absorbency material commonly known as superabsorbents. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent structure to better contain and absorb body exudates. The concentration of the superabsorbent particles may also vary through the thickness of the absorbent structure. Alternatively, the absorbent structure may comprise a laminate of fibrous webs and superabsorbent material or other suitable means of maintaining a superabsorbent material in a localized area.

The absorbent structure 38 can have any of a number of shapes. For example, the structure can be rectangular, dogbone, hourglass, I-shaped, or T-shaped. It is generally preferred that the absorbent structure be narrower in the crotch portion 26 than in the front or rear portions (22, 24) of the absorbent article.

The size and the absorbent capacity of the absorbent structure should be compatible with the size of the intended wearer and the liquid loading imparted by the intended use of the absorbent article.

The high-absorbency material can be selected from natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methylcellulose, carboxymethyl cellulose, hydroxypropyl cellulose, and the natural gums, such as alginates, xanthum gum, locust bean gum, and the like.

Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va. and DOW DRYTECH 2035 LD polymer available from Dow Chemical Co. located in Midland, Mich.

The high-absorbency material may be in any of a wide variety of geometric forms. As a general rule, it is preferred that the high-absorbency material be in the form of discrete particles. However, the high-absorbency material may also be in the form of fibers, flakes, rods, spheres, needles, or the like. As a general rule, the high-absorbency material is present in the absorbent structure 38 in an amount of from about 5 to about 90 weight percent based on total weight of the absorbent structure.

Optionally, a substantially hydrophilic tissue wrapsheet (not illustrated) may be employed to help maintain the integrity of an airlaid fibrous asorbent structure 38. The tissue wrapsheet is typically placed about the absorbent structure over at least the two major facing surfaces thereof and composed of an absorbent cellulosic material, such as creped wadding or a high wet-strength tissue. The tissue wrapsheet can be configured as a fluid intake/distribution layer 40 or another material can function is this capacity.

A fluid intake/distribution layer 40 can be placed between the absorbent structure 38 and the topsheet 42 if desired. The fluid intake/distribution layer functions to quickly uptake body exudates and discharge them into the absorbent structure 38. Suitable materials for the fluid intake/distribution layer include any woven or nonwoven web adapted to quickly take in and/or transport liquids. For example, the fluid intake/distribution layer can include a nonwoven layer of a spunbonded, a meltblown, or a bonded-carded web composed of synthetic polymer filaments or fibers, such as polypropylene, polyethylene, polyesters or the like, or a web of natural polymer filaments or fibers such as rayon or cotton. A particularly beneficial intake/distribution material is a 50.8 gsm side-by-side bicomponent polypropylene/polyethylene filament spunbond PRISM material treated with 2.25% by weight Ahcovel and SF-19 (2:1 ratio) wetting agent. PRISM is commercially available from Kimberly-Clark Corporation having offices in Neenah, Wis.

To provide improved fit and to help reduce leakage of body exudates from the absorbent article 20, the side edges 28 can be elasticized with any suitable elastic member, such as single or multiple strands of elastic. The elastic strands can be composed of natural or synthetic rubber and can optionally be heat shrinkable or heat elasticizable material. The leg elastic members 44 are constructed to gather and shirr the side edges 28 to provide elasticized leg bands which can closely fit around the legs of the wearer to reduce leakage and provide improved comfort and appearance. The leg elastic members 44 can extend along the entire length of the leg cutout 31 or along only a portion thereof. Generally, the leg elastic members are positioned adjacent the side edges 28, but they can be placed at the side edges, or wrap around the side edges. The leg elastic members can comprise a single elastic member or several elastic members operatively associated together.

It will be appreciated that the foregoing background, summary, and detailed description of the invention are given for the purposes of illustration, and as such are not intended to be construed as limiting the scope of the invention, which is defined by the following claims and all equivalents thereto.

We claim:

1. An absorbent article comprising:
a front portion having a front center region, a pair of front side regions located adjacent the front center region, and a pair of front side edges, the front center and the front side regions each having a plurality of zigzag elastic members, wherein the stretch of the front center region is less than the stretch of the front side elastic regions when subjected to the same tensile force;
a back portion having a pair of back side edges;
a crotch portion having an absorbent structure connecting the front and back portions; and
a bottomsheet secured to at least one of the front, back, or crotch portions.

2. The absorbent article of claim 1 wherein the front, back, and crotch portions are integral and formed from at least one unitary sheet.

3. The absorbent article of claim 1 wherein at least two of the front, back, or crotch portions are separate pieces joined to each other.

4. The absorbent article of claim 1 wherein the front center portion and the front side portions are separate pieces joined to each other.

5. The absorbent article of claim 1 wherein the plurality of zigzag elastic members in the front center and the front side regions comprise three separate non-overlapping elastic regions.

6. The absorbent article of claim 1 wherein the plurality of zigzag elastic members in the front side regions overlap the front center region.

7. The absorbent article of claim 1 wherein the back portion further comprises a back center region and a pair of back side regions, the back center region and the back side regions each having a plurality of zigzag elastic members wherein the stretch of the back center region is less than the stretch of the back side regions when subjected to the same tensile force.

8. The absorbent article of claim 1 further comprising a pair of leg cutouts and wherein at least a portion of the zigzag elastic members in the front side regions are tapered to follow at least a portion of the leg cutouts.

9. The absorbent article of claim 1 wherein the stretch of the front side regions is about 150 percent or greater and the stretch of the front center region is about 150 percent or less.

10. The absorbent article of claim 1 further comprising a topsheet and a pair of fasteners connected to the back side edges.

11. An absorbent article comprising:
a front portion having a front center region, a pair of front side regions located adjacent the front center region, and a pair of front side edges, the front center and the front side regions each having a plurality of zigzag elastic members, wherein the stretch of the front center region is less than the stretch of the front side regions when subjected to the same tensile force;
a back portion having a back center region, a pair of back side regions located adjacent the back center region, and a pair of back side edges, the back center and the back side regions each having a plurality of zigzag elastic members, wherein the stretch of the back center region is less than the stretch of the back side regions when subjected to the same tensile force;
a crotch portion having an absorbent structure connecting the front and back portions;
a bottomsheet secured to at least one of the front, back, or crotch portions; and
the front side edges joined to the back side edges forming an underwear like absorbent article having a waist opening and a pair of leg openings.

12. The absorbent article of claim 11 wherein the plurality of zigzag elastic members in the front center, front side regions, back center, and back side regions comprise six separate non-overlapping elastic regions.

13. The absorbent article of claim 11 wherein the plurality of zigzag elastic members in the front side regions overlap the front center region, and the plurality of zigzag elastic members in the back side regions overlap the back center region.

14. The absorbent article of claim 11 wherein the elastic members in the front side regions and the back side regions form an arcuate curve pointing towards the front and back center regions.

15. The absorbent article of claim 11 wherein the zigzag elastic members in the front center region are applied in an elongated oval and the zigzag elastic members in the back center region are applied in a rectangular pattern.

16. An absorbent article comprising:
a front portion having a front center region, a pair of front side regions located adjacent the front center region, and a pair of front side edges, the front center and the front side regions each having a plurality of zigzag elastic members, wherein the stretch of the front center region is less than the stretch of the front side elastic regions when subjected to the same tensile force;
a back portion having a back center region, a pair of back side regions located adjacent the back center region, and a pair of back side edges, the back center and the back side regions each having a plurality of zigzag elastic members, wherein the stretch of the back center region is less than the stretch of the back side regions when subjected to the same tensile force;
a crotch portion having an absorbent structure connecting the front and back portions;
a bottomsheet secured to at least one of the front, back, or crotch portions; and
the front side edges connected to the back side edges by a pair of refastenable members and by a pair of semi-permanent bonds forming an underwear like absorbent article having a waist opening and a pair of leg openings, and wherein the absorbent article is capable of being adjusted or removed by breaking the semi-permanent bonds.

17. The absorbent article of claim 16 wherein the stretch of the front and back side regions is about 200 percent or greater and the stretch of the front and back center regions is about 100 percent or less.

18. The absorbent article of claim 16 wherein the stretch of the front and back side regions is about 200 percent or greater and the stretch of the front and back center regions is about 50 percent or less.

19. The absorbent article of claim 16 wherein the plurality of zigzag elastic members in the front center, front side regions, back center, and back side regions comprise six separate non-overlapping elastic regions.

20. The absorbent article of claim 16 wherein the plurality of zigzag elastic members in the front side regions overlap the front center region, and the plurality of zigzag elastic members in the back side regions overlap the back center region.

21. The absorbent article of claim 16 wherein the elastic members in the front side regions and the back side regions form an arcuate curve pointing towards the front and back center regions.

22. The absorbent article of claim 16 wherein the zigzag elastic members in the front center region are applied in an elongated oval and the zigzag elastic members in the back center region are applied in a rectangular pattern.
